(19) United States
(12) Patent Application Publication
(10) Pub. No.: US 2018/0348751 A1
(43) Pub. Date: Dec. 6, 2018

(54) Partially Autonomous Vehicle Passenger Control in Difficult Scenario

(71) Applicant: NIO USA, Inc., San Jose, CA (US)

(72) Inventor: Austin L. Newman, San Jose, CA (US)

(73) Assignee: NIO USA, Inc., San Jose, CA (US)

(21) Appl. No.: 15/609,157

(22) Filed: May 31, 2017

(51) Int. Cl. G05D 1/00; B60W 10/20; B60W 10/18; G05D 1/02; B60W 50/14; G06K 9/00; A61B 5/18; A61B 5/024; A61B 5/00

(52) U.S. Cl. CPC G05D 1/0055; G06K 9/00892; B60W 10/18; G05D 1/0246; B60W 50/14; G06K 9/00845; A61B 5/18; A61B 5/024; A61B 5/4064; A61B 5/7405; A61B 5/742; G05D 1/0088; B60W 2050/146; B60W 2420/42; B60W 2510/20; B60W 2510/18; B60W 2710/20; B60W 2710/18; B60W 2540/22; B60W 10/20

(57) ABSTRACT

A system for autonomous driving of a vehicle having a steering system and a braking system is disclosed. The system comprises a sensor capable of gathering data relating to a driving environment, a control system programmed to control the steering system and the braking system to drive the vehicle without unrequested user intervention, in response to data gathered by the sensor, a user output device capable of presenting human readable text or speech to a user when an aspect of the driving environment detected by the sensor renders the control system unable to determine a next driving action; and, a user input device coupled to the control system. The user input device is capable of receiving user input to instruct the control system to take a user-preferred driving action. The control system is capable of instructing the steering system and the braking system to execute the user-preferred driving action.

### FIELD OF INVENTION

This application relates to autonomous vehicles. Specifically, it relates to determining when an autonomous vehicle should request input from a passenger to determine a subsequent taking a driving action, and how to obtain and handle that input.

### BACKGROUND

An autonomous vehicle includes circuitry which is programmed with decision logic that instructs the vehicle to react to road conditions while it is driving. These road conditions can include weather, traffic signs and signals, lane markers, and other vehicles, pedestrians or objects in the road. These road conditions are detected via detection hardware such as cameras, microphones, sensors, and other hardware known in the art to measure aspects of the surrounding environment such as the presence or absence of objects, the color of a signal, or the wording of a sign. Factors that comprise the driving environment are fed through logic hardware, e.g. a microprocessor programmed to analyze the driving environment and decide what actions to take next. Detection hardware analyzes the environment, which is constantly changing as the vehicle, and other vehicles, in traffic, move along the road and as traffic signals change.

The driving environment can inform the vehicle's decision logic, and could cause the vehicle to respond to changes in the environment by executing a driving action. Driving actions can include acceleration, deceleration, braking, turning, swerving, or other actions a driver of a vehicle that was not autonomous might execute with the ordinary interface elements of a vehicle, e.g. a steering wheel, gas pedal, brake pedal, gear shift, turn signal, etc.

In certain instances, the vehicle's decision logic might not be programmed with a preferred response to a particular change in the driving environment, because such a change may be unfamiliar or unanticipated. For example, another vehicle in the road may be exhibiting unpredictable driving behavior.

It would be desirable to provide a system that can seek input from a passenger in the event of such unexpected events while maintaining the autonomous driving of the vehicle during the times when such unexpected events are not occurring.

### SUMMARY

In one aspect, a system for autonomous driving of a vehicle having a steering system and a braking system is disclosed. The system comprises a sensor capable of gathering data relating to a driving environment, a control system programmed to control the steering system and the braking system to drive the vehicle without unrequested user intervention, in response to data gathered by the sensor, a user output device capable of presenting human readable text or speech to a user when an aspect of the driving environment detected by the sensor renders the control system unable to determine a next driving action; and, a user input device coupled to the control system. The user input device is capable of receiving user input to instruct the control system to take a user-preferred driving action. The control system is capable of instructing the steering system and the braking system to execute the user-preferred driving action.

### BRIEF DESCRIPTION OF THE DRAWING(S)

FIG. 1 is a schematic of an autonomous vehicle including sensors.

FIG. 2 is a block diagram of the control system of an autonomous vehicle.

FIG. 3 is a flow diagram of a method of requesting, receiving and responding to passenger input during autonomous driving.

### DETAILED DESCRIPTION OF THE PREFERRED EMBODIMENT(S)

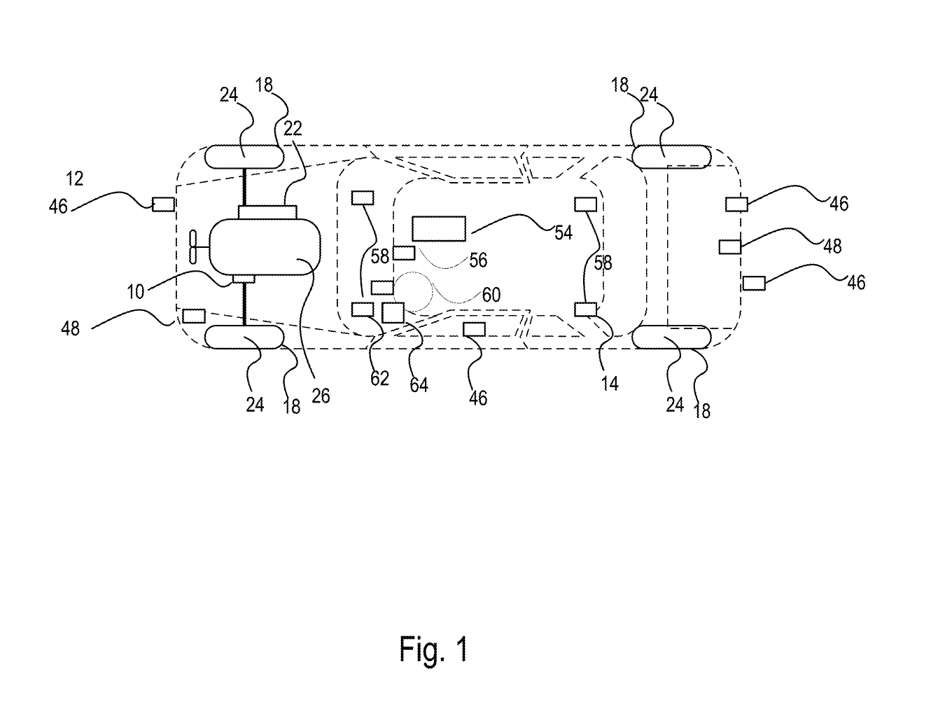

FIG. 1 is a schematic of an autonomous vehicle 12. Vehicle 12 comprises wheels 24 and an engine 26. Wheels 24 are coupled to the engine 26, such that the engine 26 supplies power to rotate the wheels 24. Wheels 24 are also coupled to brakes 18 which allow the vehicle to stop.

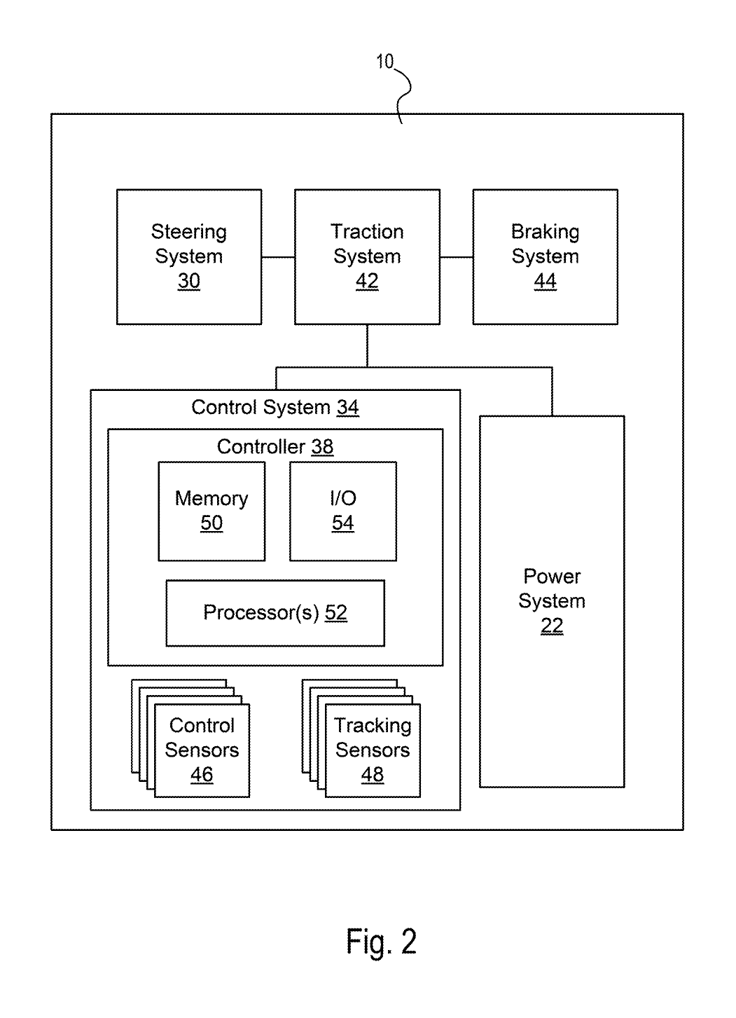

Vehicle 12 also comprises a control system 10, which is coupled to the engine 26. A block diagram of the control system 10 is shown in FIG. 2. Control system 10 comprises a steering system 30, which is coupled to the wheels 24 to determine their orientation at any given time, allowing the vehicle 12 to be steered. Control system 10 further comprises a traction system 42 and a braking system 44, which together control the wheels 24 and the brakes 18. The traction system 42 is coupled to a power system 22 and a control system 34. Power system 22 supplies power to the engine 26, the control system 34, and all other powered components needed to power the vehicle 12. Power system 22 can run on electricity, e.g., supplied by a battery, and/or can run on a fuel such as gasoline.

Control system 34 comprises a memory 50, a processor 52, and an input/output (“I/O”) controller 54. Memory 50 comprises machine readable code that includes instruction logic, which is programmed to instruct the control system 34 to, e.g., react to data received via the I/O interface 54 and choose and execute a driving instruction. Control system 34 further comprises control sensors 46 and tracking sensors 48, which are strategically placed inside and outside the vehicle 12. Control sensors 46 and tracking sensors 48 can include cameras to sense aspects of the driving environment. Data from the control sensors 46 and tracking sensors 48 is fed to the controller 38 via the I/O interface 54. The processor 50, using the machine readable code stored in the memory 54, analyzes the data from the control sensors 46 and tracking sensors 48 and chooses a driving action that the vehicle should take. The control system 34 then sends instructions to the steering system 30, the tracking system 42 and the braking system 44 to execute the driving action.

For instance, a control sensor 46 comprising a camera may detect the presence of a traffic light with the red light activated. Tracking sensor 48 may also sense the location of a white line in the road, the so-called “stop line,” behind which the law requires vehicles to stop when the traffic light is red. The presence of a red light, detected by the control sensors 46 and the tracking sensors 48, is fed to the controller 38, which, based on the logic in the machine readable code stored in the memory, determines that the vehicle 12 must stop at the stop line. The control system 34 then instructs the braking system 44 to stop when the control sensors 46 and tracking sensors 48 indicate that the vehicle 12 has reached the stop line.

In certain instances, the machine readable code stored in memory 50 might not be programmed with a preferred response to a particular change in the driving environment, because such a change may be unfamiliar or unanticipated. For example, another vehicle in the road may be exhibiting unpredictable driving behavior, or there may be an object in or near the road, or a traffic sign or signal that is detected by control sensors 46 or tracking sensors 48 but that the system does not recognize.

Turning back to FIG. 1, in one aspect, the vehicle 12 is equipped with user interface equipment, such as a touch screen 54, where the passenger is presented with options via the touch screen 54, so the passenger may choose how to proceed from a series of choices. Alternatively or additionally, the vehicle could be equipped with a microphone 56 and speakers 58, which can be used for voice recognition. Accordingly, the control system 34 would present the passenger with options, either via a screen such as touch screen 54 or aurally via speakers 58. The passenger could respond by speaking. The passenger's spoken words would be recorded by microphone 58 and processed by control system 34 via I/O 54. Other user input and user output devices could also be used. In response, control system 34 would instruct the steering system 30, the traction system 42 and braking system 44 to undertake the driving action chosen by the passenger (e.g. “pull over” or “stop.”)

Alternatively or in addition to touch screen 54 or voice prompts, the vehicle may be equipped with standard automobile inputs, such as a steering wheel 60, gas pedal 62, and brake pedal 64, etc. In one aspect, the control system 34 can instruct the passenger, via a screen such as touch screen 54 or via speakers 58, to take over driving using the traditional automobile inputs, when the control system 34 detects a change in the driving environment for which control system 34 does not have a programmed response.

Control sensors 46 may also include biometric sensors, which may be coupled to one or more passengers while the vehicle 12 is driving. A passenger in a vehicle may be more acutely aware of a potentially important or dangerous change in the driving environment despite the presence of control sensors 46 and tracking sensors 48, even if the system is not detecting the situation or if the control system 34, executing the machine readable code stored in memory 50 and executed by processor 52 in response to input from control sensors 46 and tracking sensors 48, cannot decipher the situation or does not have the confidence necessary to decide that a particular driving action is the correct one. The passenger may manifest that awareness in objective biometric changes that could be detectable by biometric sensors in the vehicle, such as those that can detect, e.g., relevant changes in heart rate, blood pressure, the pace of breathing, perspiration, brain activity, or other biometric indicators that an emergent situation on the road might need to be dealt with via input from the passenger.

When the vehicle's decision logic detects these changes in the biometrics of the passenger, the vehicle can seek the user's input regarding what to do next, via the touch screen 54 or speakers 58. As recited above, the passenger would respond to the request of the control system 34 for an instruction via touch screen 54 or via speaking into microphone 56. The user's input is handled via I/O 54 and the user's instruction is sent from control system 34 to steering system 30, traction system 42 and braking system 44. Alternatively, the control system 34 can instruct the passenger to take over driving via traditional automobile inputs such as steering wheel 60.

In one aspect, the memory 50 of control system 34 may contain machine readable code which permits the storage of data relating to the driving environment along with the user's response. The control system 34 may use this information to learn from a passenger's input and the observed environmental factors, so that the system might be able to handle a similar situation in the future without user intervention.

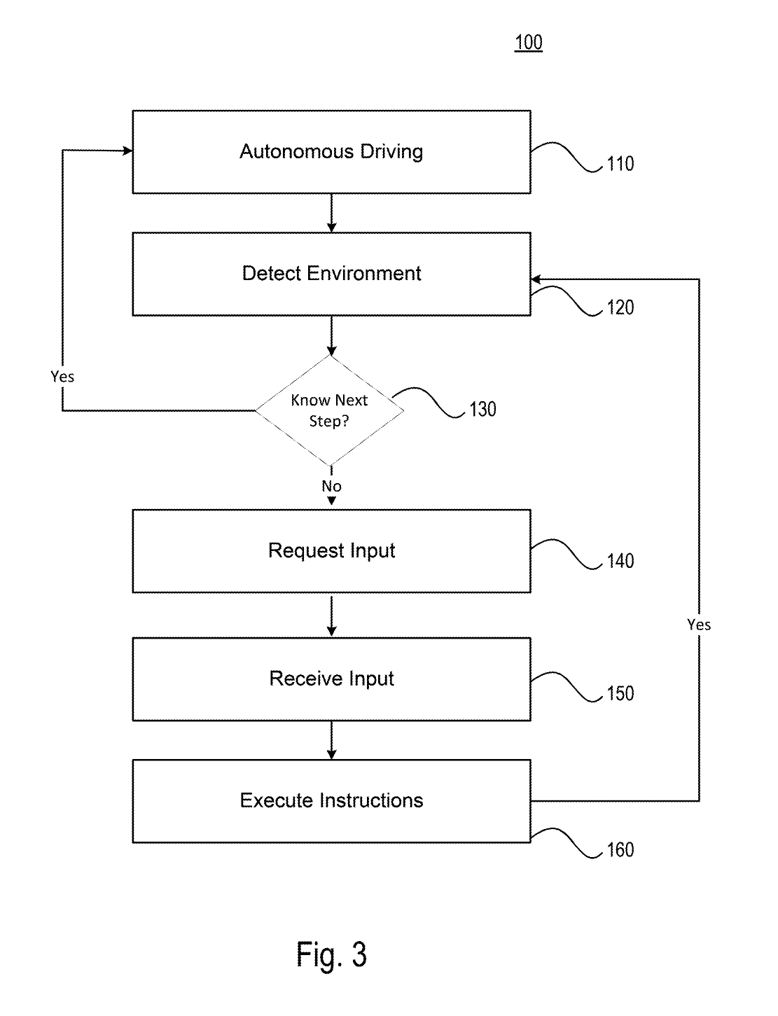

FIG. 3 is a flow chart of a process 100 for determining whether user input is needed, and receiving such input. In step 110, the autonomous vehicle is driving without user intervention. The autonomous vehicle periodically detects the driving and passenger environment in step 120. This detection is done using control sensors 46 and tracking sensors 48 to send data via I/O 56 to control system 34. Control system 34 instructs steering system 30, traction system 42 and braking system 44 in response to data received from sensors 46 and 48. In step 130, after an environmental variable is detected, the control system 34 determines whether it knows what the next driving action should be, based on the environmental variables detected. Environmental variables can include variables about the driving environment such as road conditions or traffic signals, or can also include data from the biometric sensors that indicate that the passenger is responding to an emergency situation that the other sensors have not detected.

If the control system 34 knows, with sufficient confidence, what the next driving action should be, it returns to step 110 and continues autonomous driving until the instance of step 120 wherein the environment is scanned for changes. If, after scanning the environment, the control system 34 does not know, with sufficient confidence, what the next driving step should be, the system requests input from the user at step 140.

As discussed above, the request can be in the form of words appearing on a screen or words spoken through speakers, or any other way of alerting the passenger to pay attention, take action, and/or to answer a question or a request. In step 150, the system receives user input. As discussed, the input can be in the form of a chosen multiple choice answer on a touch screen, a voice command, or the passenger can begin driving the vehicle using traditional driving controls such as a steering wheel and an accelerator pedal. The control system 34 then receives the user input and, in step 160, instructs the steering system 30, traction system 42, and braking system 44 to execute the passenger's command. Finally, the system returns to step 120 to detect the environment again and determines in step 130 whether it needs further user input or can resume autonomous driving.

Having thus described the presently preferred embodiments in detail, it is to be appreciated and will be apparent to those skilled in the art that many physical changes, only a few of which are exemplified in the detailed description of the invention, could be made without altering the inventive concepts and principles embodied therein. It is also to be appreciated that numerous embodiments incorporating only part of the preferred embodiment are possible which do not alter, with respect to those parts, the inventive concepts and principles embodied therein. The present embodiments and optional configurations are therefore to be considered in all respects as exemplary and/or illustrative and not restrictive, the scope of the invention being indicated by the appended claims rather than by the foregoing description, and all alternate embodiments and changes to this embodiment which come within the meaning and range of equivalency of said claims are therefore to be embraced therein.

What is claimed is:

1. A system for autonomous driving of a vehicle having a steering system and a braking system, the system comprising:
a sensor capable of gathering data relating to a driving environment;
a control system programmed to control the steering system and the braking system to drive the vehicle without unrequested user intervention, in response to data gathered by the sensor;
a user output device capable of presenting human readable text or speech to a user when an aspect of the driving environment detected by the sensor renders the control system unable to determine a next driving action; and
a user input device coupled to the control system, the user input device capable of receiving user input to instruct the control system to take a user-preferred driving action, wheren the control system is capable of instructing the steering system and the braking system to execute the user-preferred driving action.

2. The system of claim 1, wherein the sensor comprises a camera.

3. The system of 1, wherein the sensor comprises a biometric sensor capable of obtaining biometric data from the user.

4. The system of claim 3, wherein the driving environment comprises biometric data indicating that the user perceives an emergency.

5. The system of claim 3, wherein the biometric data relates to heart rate.

6. The system of claim 3, wherein the biometric data relates to brain activity.

7. The system of claim 1, wherein the user output device is a speaker.

8. The system of claim 1, wherein the user output device is a screen.

9. The system of claim 1, wherein a touch screen is both the user input device and the user output device, and wherein the user input is a response to a multiple choice question displayed on the touch screen.

10. A method of autonomous driving of a vehicle having a steering system and a braking system, the method comprising:
gathering data relating to a driving environment via a sensor;
controling the steering system and the braking system, via a control system, to drive the vehicle without unrequested user intervention, in response to data gathered by the sensor;
presenting human readable text or speech to a user when an aspect of the driving environment detected by the sensor renders the control system unable to determine a next driving action; and
receiving user input to instruct the control system to take a user-preferred driving action,
instructing the steering system and the braking system to execute the user-preferred driving action.

11. The method of claim 10, wherein the sensor comprises a camera.

12. The method of 10, further comprising obtaining biometric data from the user.

13. The method of claim 12, wherein the driving environment comprises biometric data indicating that the user perceives an emergency.

14. The method of claim 12, wherein the biometric data relates to heart rate.

15. The method of claim 12, wherein the biometric data relates to brain activity.

16. The method of claim 10, wherein the user output is presented via a screen.

17. The method of claim 10, wherein human readable text is a multiple choice question displayed on a touch screen, and wherein the user input is a response to the multiple choice question input via the touch screen.
